(19) United States
(12) Patent Application Publication
(10) Pub. No.: US 2023/0364034 A1
(43) Pub. Date: Nov. 16, 2023

(54) METHODS OF INHIBITING DISEASES CAUSED BY RESPIRATORY VIRUSES

(71) Applicant: Adulsa Therapeutics Corp., Berwyn, PA (US)

(72) Inventor: Leopold Galland, New York, NY (US)

(73) Assignee: Adulsa Therapeutics Corp., Berwyn, PA (US)

(21) Appl. No.: 18/026,229

(22) PCT Filed: Sep. 15, 2021
(86) PCT No.: PCT/US2021/050474

§ 371 Date: Mar. 14, 2023

Related U.S. Application Data
(60) Provisional application No. 63/079,663, filed on Sep. 17, 2020.

(51) Int. Cl. A61K 31/137; A61K 9/00; A61K 47/10

(52) U.S. Cl. CPC A61K 31/137; A61K 9/0043; A61K 47/10

(57) ABSTRACT

The present invention provides a method of inhibiting a viral respiratory disease, comprising: applying to the nasal lining of a subject, in need thereof, an effective amount of a pharmaceutical composition prior to exposure to a virus, wherein the pharmaceutical composition comprises bromhexine and a solvent.

### CROSS-REFERENCE TO RELATED APPLICATIONS

This application claims the benefit of International Application No. PCT/US2021/050474, filed Sep. 15, 2021, which claims priority to the U.S. Provisional Application No. 63/079,663, filed Sep. 17, 2020, the disclosures of which are incorporated herein by reference in their entireties.

### BACKGROUND OF THE INVENTION

Coronaviruses (CoV) are a large family of viruses that cause illnesses ranging from the common cold to more severe diseases such as Middle East Respiratory Syndrome (MERS), Severe Acute Respiratory Syndrome (SARS) and Coronavirus disease 2019 (CoViD-19). Coronaviruses are zoonotic, meaning they are transmitted between animals and people, e.g., SARS-CoV from civet cats to humans; MERS-CoV from dromedary camels to humans. Several known coronaviruses are circulating in animals that have not yet infected humans.

Coronavirus Disease 2019 (CoViD-19) is a novel infectious disease caused by the Severe Acute Respiratory Syndrome-Coronavirus-2 (SARS-CoV-2). This disease has spread globally since 2019, resulting in the 2019-2020 coronavirus pandemic.

Common symptoms of CoViD-19 include fever, cough, shortness of breath, and loss of taste/smell sensations. Muscle pain, sputum production and sore throat are some of the less common symptoms. While the majority of cases result in mild symptoms, some progress to pneumonia, severe acute respiratory syndrome, multi-organ failure and even death. The case fatality rate is estimated at between 1% and 5% but varies by age and other health conditions. The disease is more likely to occur in people whose immune system is impaired due to age, immunosuppressive therapy, psychological stress, or other factors.

The infection is spread from one person to others via respiratory droplets, often produced during coughing. Time from exposure to onset of symptoms is generally between two and fourteen days, with an average of five days. The standard method of diagnosis is by reverse transcription polymerase chain reaction (rRT-PCR) from a nasopharyngeal swab or sputum sample. Antibody assays are performed using a blood serum sample. The infection can also be diagnosed from a combination of symptoms, risk factors and a chest CT scan showing features of pneumonia.

Because a vaccine against SARS-CoV-2 is not expected to become available until 2021, at the earliest, a key part of managing the CoViD-19 pandemic is by trying to decrease the epidemic peak (i.e., preventing transmission of CoViD-19), known as flattening the epidemic curve. Such helps decrease the risk of health services being overwhelmed and provides more time for a vaccine and treatment to be developed. Recommended measures to prevent the transmission of CoViD-19 include frequent hand washing, maintaining distance from other people, not touching one's face, covering mouth/nose when coughing/sneezing, and the use of masks. CoViD-19 is a poorly understood disease with unique clinical/transmission characteristics, evinced by global health experts who provide daily updates with varying medical advice.

Accordingly, an urgent need exists for an effective and safe method of preventing and/or inhibiting CoViD-19, and the symptoms thereof.

### SUMMARY OF THE INVENTION

In one aspect, the present invention provides methods of inhibiting a viral respiratory disease. The methods comprise applying to the nasal lining of a subject, in need thereof, an effective amount of a pharmaceutical composition prior to exposure to a virus. The pharmaceutical composition comprises bromhexine (e.g., bromhexine hydrochloride, bromhexine chloride, or mixtures thereof) and a solvent (e.g., alcohol and/or water). Examples of alcohol include xylitol, polyethylene glycol, propylene glycol or mixtures thereof. In one embodiment, the respiratory disease is caused by a corona virus or influenza virus. In one embodiment, the corona virus is an alpha coronavirus, a beta coronavirus, MERS-CoV, SAR-CoV or SARS-CoV-2. In one embodiment, the disease is the common cold, MERS, SARS or CoViD-19. In one embodiment, the bromhexine applied via a nasal spray to both nostrils in a dose of about 0.1 mg to about 0.7 mg. In one embodiment, the amount of bromhexine applied to each nostril is about 1.2 mg per day.

### DETAILED DESCRIPTION OF THE INVENTION

The methods of the present invention provide prophylactic treatment and inhibition of contracting respiratory diseases. The methods include topically applying pharmaceutical compositions to the nasal lining of mammals, typically humans. Examples of respiratory diseases are diseases caused by corona viruses and influenza viruses.

Examples of corona viruses include SARS-CoV-2, SAR-CoV, MERS-CoV, the alpha coronaviruses (e.g., 229E, NL63), and the beta coronaviruses (e.g., 0C43, HKU1). SARS-CoV-2 causes Coronavirus Disease 2019 (i.e., CoViD-19). SARS-CoV causes Severe Acute Respiratory Syndrome (i.e., SARS). MERS-CoV causes Middle East Respiratory Syndrome (i.e., MERS). The alpha and beta coronaviruses usually cause mild to moderate upper-respiratory tract illnesses, like the common cold.

Examples of Influenza Viruses include Swine Influenza Viruses (SIVs) and Avian Influenza Viruses. The known SW strains include Influenza C and the subtypes of Influenza A known as H1N1, H1N2, H3N1, H3N2 and H2N3. Influenza A virus subtype H1N1 (A/H1N1) was the most common cause of human influenza in 2009, and is associated with the 1918 outbreak known as the Spanish flu. An emerging Avian Influenza Virus is the Highly Pathogenic Avian Influenza A virus subtype H5N1. Since the first human H5N1 outbreak in 1997, there has been an increasing number of HPAI H5N1 bird-to-human transmissions, leading to clinically severe and fatal human infections.

The methods of the instant invention comprise the topical application of a pharmaceutical composition to the nasal lining of a mammal, typically a human, in an amount which is effective to inhibit diseases caused by respiratory viruses. Examples of other mammals include pets (e.g., dogs, cats, etc.), livestock (e.g., cows, cattle, pigs, etc.) and laboratory animals (e.g., rodents, primates, etc.). Inhibition of diseases includes prevention of a disease, and inhibition of any symptom associated with a disease.

The pharmaceutical composition comprises bromhexine formulated for topical application to the nasal lining. In one embodiment, bromhexine comprises bromhexine hydrochloride, bromhexine chloride, or mixtures thereof. In one embodiment, the pharmaceutical composition comprises a pharmaceutical carrier/excipient, such as, for example, an alcohol (e.g., xylitol, polyethylene glycol, propylene glycol, glycerin and sorbitol) and/or water. The bromhexine is dissolved in the carrier/excipient. For example, bromhexine is dissolved in a solvent, which solvent comprises an alcohol and water. Typically, the alcohol comprises about 5-25% of the solvent, more typically about 11-20% of the solvent.

The pharmaceutical composition is formulated in such a manner to minimize systemic absorption of the bromhexine, preferably to completely avoid systemic absorption, thereby avoiding systemic effects. The composition would remain localized to the cells lining the inside of the nose. Thus, bromhexine would not reach the lungs. Consequently, the composition would not have any effect on any viral load that may be in the system. Such avoidance of any systemic effect is accomplished by providing a minimum dose of bromhexine.

In one embodiment, the pharmaceutical composition is formulated as a nasal spray or nasal mist or nasal drops. The bromhexine in the carrier solvent has a concentration of about 0.5 to 5 mg/ml, more preferably about 1 to 2.5 mg/ml, even more preferably about 2 mg/ml. A typical dose of the bromhexine is about 0.1 mg to about 0.7 mg, more typically, about 0.3 mg to about 0.5 mg, even more typically about 0.4 mg. The spray is typically applied about 1 to 4 times a day, preferably 3 times a day into each nostril. The total daily dose of bromhexine applied into each nostril is about 0.1 to 2.8 mg per day, typically about 0.5 to 2.0 mg per day, more typically about 0.8 to 1.8 mg per day, most typically, about 1.2 mg per day. Preferably, the bromhexine is applied to both nostrils.

In one embodiment, the pharmaceutical composition is formulated as a gel. Examples of a gel carrier include propylene glycol and glycerin, and optionally, including additional thickening agents. The bromhexine in the carrier solvent has a concentration of about 0.5 to 5 mg/ml, more preferably about 1 to 2.5 mg/ml, even more preferably about 2 mg/ml. A typical dose of the bromhexine is about 0.1 mg to about 0.7 mg, more typically, about 0.3 mg to about 0.5 mg, even more typically about 0.4 mg. The gel is typically applied about 1 to 4 times a day, preferably 3 times a day into each nostril. The total daily dose of bromhexine applied to each nostril is about 0.1 to 2.8 mg per day, typically about 0.5 to 2.0 mg per day, more typically about 0.8 to 1.8 mg per day, most typically, about 1.2 mg per day. Preferably, the bromhexine is applied to both nostrils.

Notwithstanding the foregoing, it is noted that the actual preferred amounts of a pharmaceutical composition in a specific case will vary according to the particular compositions formulated, the mode of application, and the subject being treated (e.g., age, gender, size, tolerance to drug, etc.), as would be known to a skilled artisan with the foregoing guidance.

Without wanting to be limited to a mechanism of action, it is believed that the nasal lining cells (i.e., the epithelium) are the principal site of entry of a respiratory virus into the body; and that bromhexine topically applied to the nasal epithelium prevents cellular entry of the virus. In particular, it is believed that respiratory viruses bind to a protein in the cell membrane called the cellular receptor (e.g., the ACE2 cellular receptor), and use the serine protease TMPRSS2 (i.e., transmembrane protease, serine 2) to enter the cell. The compositions of the present invention inhibit the TMPRSS2 in the nasal lining, thereby blocking entry of the respiratory viruses. Thus, it is important that the compositions of the present invention be applied to both nostrils since entry should be prevented from either nostril.

In the present specification, bromhexine includes all pharmaceutically acceptable versions thereof, including, for example, all pharmaceutically acceptable salts thereof, stereoisomers and/or any mixtures thereof, all pharmaceutically acceptable zwitterions and/or any mixtures thereof, all pharmaceutically acceptable polymorphic forms and/or any mixtures thereof, and all pharmaceutically acceptable complexes (including solvates) and/or any mixtures thereof. Salts include their racemates, enantiomers, or any mixtures thereof. Particularly suitable salts comprise anionic minerals e.g., chlorides and hydrochlorides). Salts also include all enantiomeric salts formed with pharmaceutically acceptable chiral acids and/or bases and/or any mixtures of enantiomers of such salts (+) tartrates, (−) tartrates and/or any mixtures thereof including racemic mixtures).

In one embodiment of the present invention, the pharmaceutical compound is administered prophylactically (i.e., before exposure to a virus) to an individual who is at risk of developing a respiratory disease. Individuals who are at risk have one or more of the following conditions: are going out in public, particularly, to an environment where social distancing is not practiced or not practical (e.g., plane); are elderly (e.g., over age 65 years old); have heart disease; have lung disease (e.g., asthma); have diabetes; are obese; are smokers; are under stress and/or are depressed; have weakened immune systems; are immunocompromised; have been in contact with a person who is infected, or suspected of being infected, with a viral respiratory disease. Individuals who have weakened immune systems include, for example, cancer patients, in particular those who are undergoing chemotherapy; individuals with transplants; individuals who take pharmaceuticals to suppress their immune response (e.g., corticosteroids, cyclosporine); individuals with HW or AIDS; diabetics; individuals recovering from an illness, surgery or trauma; and individuals with poor nutrition. Prophylactic administration of the pharmaceutical composition is also suited for individuals who cannot tolerate (or cannot benefit from) a vaccine, or for whom vaccines are contraindicated. For example, it is known that many elderly individuals do not react efficiently to vaccines. Prophylactic administration of the pharmaceutical composition can be, for example, daily administration at least a few days before exposure, or right before exposure.

In another embodiment, the pharmaceutical composition is administered as soon it is first suspected that the individual was exposed to a respiratory virus, but before the onset of a symptom of the disease. Examples of early symptoms include respiratory symptoms, fever, cough, shortness of breath, breathing difficulties, muscle/joint pain and the loss of the sensation of taste/smell.

The pharmaceutical compositions of the present invention can further comprise one or more pharmaceutically acceptable additional ingredient(s) such as alum, stabilizers, buffers, coloring agents, flavoring agents, and the like.

In one embodiment, the present invention includes the pharmaceutical compositions used in the methods of the invention.

In one embodiment, the pharmaceutical composition consists essentially of: a) bromhexine; and b) a solvent. Any other ingredients which may materially affect the basic and novel characteristics of the invention are specifically excluded from the composition. For example, any ingredient (e.g., excipient or carrier) that is used for systemic administration, or would enhance systemic absorption (e.g., permeation enhancers) are specifically excluded from the compositions. For instance, in some embodiments, the following ingredients may be specifically excluded from the pharmaceutical composition of the present invention: nafamostat mesilate, aprotinin, bacitracin, soybean trypsin inhibitor, phosphoramidon, leupeptin, bestatin, surfactants (e.g., sodium taurocholate, sodium deoxycholate sodium, glycodeoxycholat, fatty acid derivatives, phospholipids), cationic polymers (e.g., chitosan and derivatives), enzyme inhibitors (e.g., human neutrophil elastase inhibitor), cyclodextrins, tight junction modulators, Sodium Benzoate, a flavoring, Tartaric Acid, Carboxymethylcellulose Sodium, Sodium Hydroxide, decongestants (e.g., naphazoline, phenylephrine, oxymetazoline and pseudoephedrine), antihistamines, corticosteroids and cromolyn. Also, any ingredient which can potentially cause an undesirable effect/side effect, including, for example, an allergic response, may be specifically excluded from the pharmaceutical compositions.

1. A method of inhibiting a viral respiratory disease, comprising:
applying to the nasal lining of a subject, in need thereof, an effective amount of a pharmaceutical composition prior to exposure to a virus, wherein the pharmaceutical composition comprises bromhexine and a solvent,
wherein the viral respiratory disease is inhibited.

2. The method according to claim 1, wherein the bromhexine comprises bromhexine hydrochloride, bromhexine chloride, or mixtures thereof; and the solvent comprises alcohol and water.

3. The method of claim 2 wherein the alcohol comprises xylitol, polyethylene glycol, propylene glycol or mixtures thereof.

4. The method according to claim 1, wherein the disease is caused by a corona virus or influenza virus.

5. The method according to claim 4, wherein the corona virus is selected from the group consisting of alpha coronaviruses, the beta coronaviruses, MERS-CoV, SAR-CoV, and SARS-CoV-2.

6. The method according to claim 5, wherein the disease is selected from the group consisting of the common cold, MERS, SARS and CoViD-19.

7. The method according to claim 1, wherein the bromhexine is applied via a nasal spray to both nostrils in a dose of about 0.1 mg to about 0.7 mg.

8. The method according to claim 7, wherein the dose of bromhexine applied to each nostril is about 1.2 mg per day.

9. The method according to claim 1, wherein the bromhexine is applied via a gel to both nostrils in a dose of about 0.1 mg to about 0.7 mg.

10. The method according to claim 9, wherein the amount of bromhexine applied to each nostril is about 1.2 mg per day.
